(19) United States
(12) Patent Application Publication
(10) Pub. No.: US 2006/0184053 A1
(43) Pub. Date: Aug. 17, 2006

(54) Tunnel type sphygmomanometer assembly

(75) Inventor: Paul Yang, Chung Ho City (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD., CHUNG HO CITY (TW)

(21) Appl. No.: 11/057,228

(22) Filed: Feb. 15, 2005

(51) Int. Cl. A61B 5/02

(52) U.S. Cl. 600/499; 600/490

(57) ABSTRACT

The present invention discloses an improved tunnel type sphygmomanometer assembly which comprises a platform, a deformable plate disposed on the platform and an air cuff covered on the surface of the plate; one end of the plate is fixed to the platform and the other end of the plate is fixed to a transmission axle under the platform; the transmission axle is driven by a driving member; and the driving member is coupled to a control device; such that when a user's arm is placed in the air cuff and the start button is turned on, the control device will start an air pump to inflate the air cuff through an air duct, and also will start the driving member such that the driving member drives the transmission axle to rotate and coil the plate on the transmission axle along the vertical direction directly onto the transmission axle. Unlike the traditional tunnel type sphygmomanometers having a plate to be pulled horizontally when the contracted plate wraps around the user's arm, the present invention provides a chassis of smaller size.

### BACKGROUND OF THE INVENTION

1. Field of the Invention

The present invention relates to a tunnel type sphygmomanometer assembly, more particularly to an improved tunnel type sphygmomanometer assembly that pulls an air cuff by a force in the vertical direction instead of the horizontal direction and coils the air cuff directly onto a transmission axle that provides a size smaller than that of a traditional tunnel type sphygmomanometer.

2. Description of the Related Art

With reference to FIG. 1, the structure of the traditional tunnel type sphygmomanometers generally comprises a base 90; a support stand 91 disposed on one side of the base 90, a semicircular and upwardly opened frame 92 disposed on the support stand 91, an air cuff 93 disposed on the frame 92, a plastic plate 94 being deformable and having a shape corresponding to the air cuff 93 coupled to the air cuff 93, and one end of the plastic plate 94 being fixed to the frame 92 and the other end passing through the frame 92 and the support stand 91 being extended to one side of the support stand 91. Further, two parallel guide tracks 95 are disposed under the support stand 91, and one end of the guide track 95 is fixed onto a stand 96 proximate to the support stand 91, and the other end is fixed to a stand 96 at a position away from the support stand 91, and a sliding base 97 is embedded individually into the guide tracks 95, and the sliding base 97 and the plastic plate 94 are extended to couple an end under the support stand 91, and the sliding base 97 is connected to a serrated belt 98, and the serrated belt 98 is driven by a motor (not shown in the figure).

Further, the air cuff 93 is connected to a pressure sensor (not shown in the figure) by an air duct (not shown in the figure), and the pressure sensor is used to detect the pulse of blood pressure. A display device (not shown in the figure) is disposed on the other side of the base 90, and the display device is coupled to a control device through an electric circuit for displaying the result of the pulse of the measured blood pressure (not shown in the figure).

When the tunnel type sphygmomanometer is used, the user's arm is placed in the tunnel formed by the air cuff 93 first. When the user turns on the start button, the control device will start an air pump to inflate the air cuff 93 and also start the motor, such that the motor drives the serrated belt 98, and the sliding base 97 is driven by the serrated belt 98 to move in a horizontal direction away from the guide track 95 and the plastic plate 94 is driven by the sliding base 97 to gradually withdraw inward to wrap the air cuff 93 around the user's arm for measuring the user's blood pressure. The result of the measured pulse of the blood pressure is displayed on the display device. However, the guide track 95 is installed along the horizontal direction and the sliding base 97 slides on the guiding track 95, therefore when the sliding base 97 drives the plastic plate 94 to move, the plastic plate 94 will also move in the horizontal direction. Therefore, it is necessary to have a sliding distance along the horizontal direction to accommodate the sliding base 97, which will cause a larger size to the sphygmomanometer.

### SUMMARY OF THE INVENTION

In view of the foregoing shortcomings of the prior art, the inventor of the present invention focused on the problem to start finding a way for the improvement and overcome the shortcomings in hope of finding a feasible solution, and conducted extensive researches and experiments and finally invented the tunnel type sphygmomanometer assembly in accordance with the present invention to reduce the size of the traditional tunnel type sphygmomanometers.

Therefore, it is the primary objective of the present invention to provide an improved tunnel type sphygmomanometer assembly, which comprises a platform, a support frame disposed under the platform, a retaining base disposed under the support frame, a transmission axle being pivotally coupled to the retaining base and driven by a driving member, and the driving member being coupled to a control device. Further, the platform comprises a deformable and coiled circular plate, an air cuff wrapped around the surface of the plate, and one end of the plate being fixed onto the platform and the other end being fixed to the transmission axle under the platform, and the transmission axle being driven by a driving member, so that when a user's arm is placed in the air cuff for measuring blood pressure, the control device will start an air pump to inflate the air cuff through an air duct, and the control device also will start the driving member, and the driving member will drive the transmission axle to rotate as to coil the plate of the transmission axle directly onto the transmission axle. As a result, the tunnel type sphygmomanometer of the present invention has a smaller size than the general prior-art tunnel type sphygmomanometer that pulls the plate along the horizontal direction.

### BRIEF DESCRIPTION OF THE DRAWINGS

The above objects, features and advantages of the present invention will become apparent from the following detailed description taken with the accompanying drawing.

FIG. 1 is a perspective view of the tunnel type sphygmomanometer assembly according to a prior art.

FIG. 2 is a perspective view of the tunnel type sphygmomanometer assembly according to the present invention.

FIG. 3 is an exploded view of the tunnel type sphygmomanometer assembly according to the present invention.

FIG. 4 is a perspective view of the plate being coupled to the transmission axle according to the present invention.

FIG. 5 is an illustrative view of the movement of the tunnel type sphygmomanometer assembly according to a preferred embodiment of the present invention.

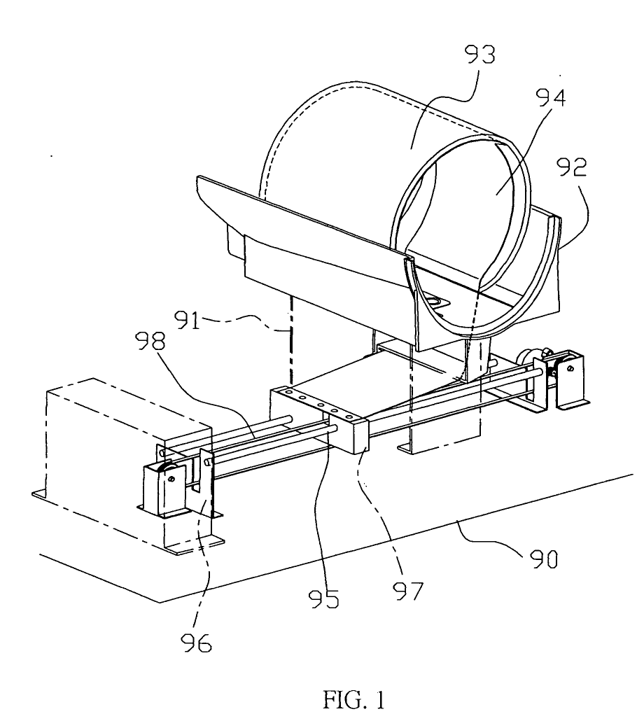

### DETAILED DESCRIPTION OF THE PREFERRED EMBODIMENTS

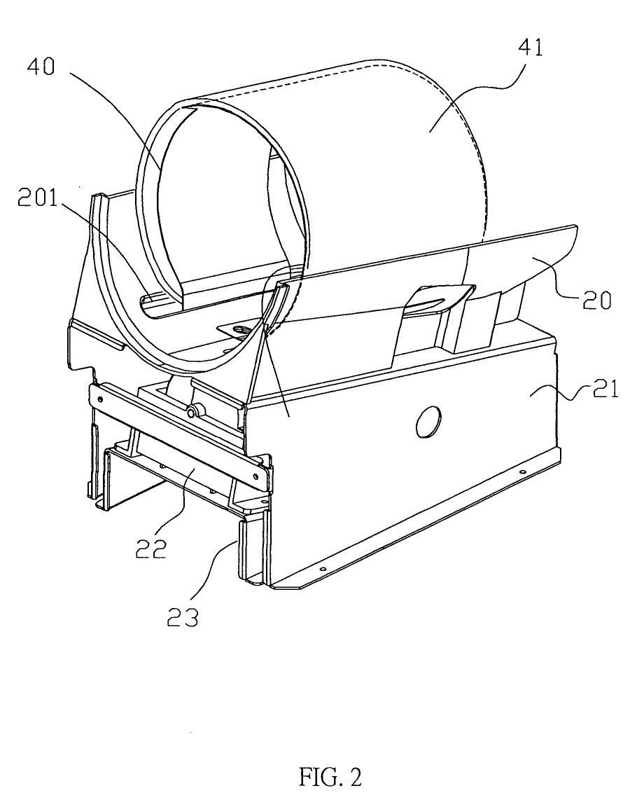

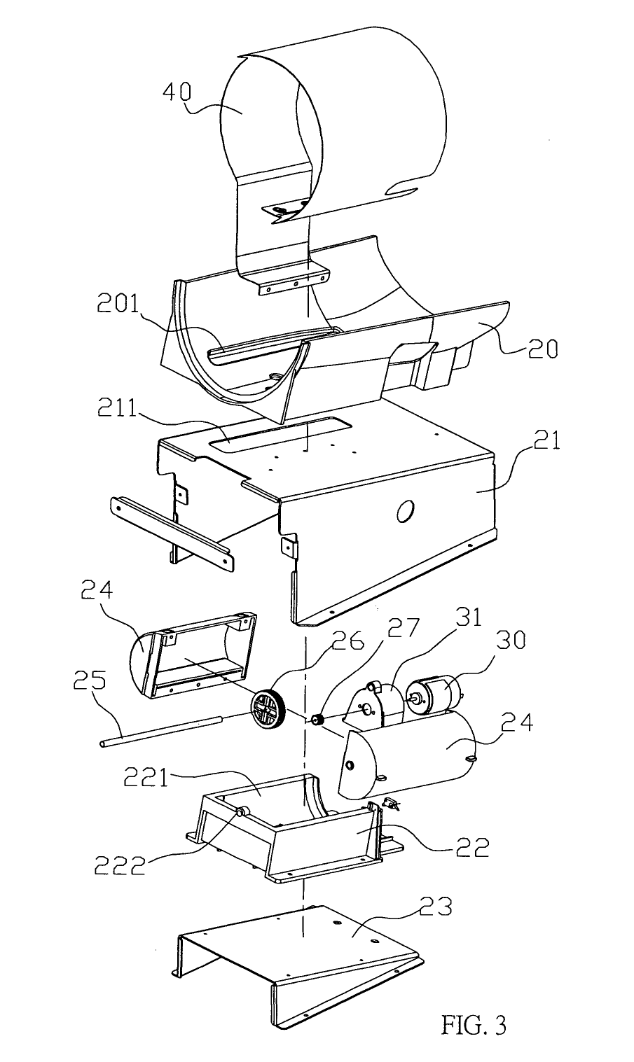

Please refer to FIGS. 2 and 3 for an improved tunnel type sphygmomanometer assembly according to the present invention. The sphygmomanometer comprises a base, a chassis disposed at an upper casing of the base, a control device, an air pump, an air duct, a sensor and a display device (not shown in the figure because these devices are the same as those used in a prior-art tunnel sphygmomanometer and not the key characteristics of this invention) being disposed on one side of the chassis, a platform 20 disposed on the other side of the chassis. The platform 20 is a semicircular upwardly opened chassis, and an aslant support frame 21 is disposed in the chassis under the platform 20. The support frame 21 has an opening 211 on one side, and a retaining base 22 under the support frame 21. The retaining base 22 has a carrying base 23 aslant towards the same direction with the support frame 21 which is disposed under the retaining base 22. The carrying base 23 is fixed onto the base.

Further, the retaining base 22 comprises a semicircular upwardly opened accommodating groove 221, and the accommodating groove 221 with a closed end comprises a pivotal hole 222, and a cylinder 24 comprised of two corresponding semicircular casings is disposed in the accommodating groove 221. One end of the cylinder 24 is a closed end, and the other end is not completely sealed but maintains a gap with respect to the opening 211 of the support frame 21, and a transmission axle 25 is disposed in the cylinder 24. The transmission axle 25 has a pivotal hole 222 pivotally coupled to one end of the transmission axle 25, and a passive gear 26 is coupled to another end of the transmission axle 25, and the passive gear 26 is engaged with an active gear 27, and the active gear 27 is mounted on a driving member 30 which is a motor in this embodiment. The driving member 30 is pivotally coupled to a driving base 31, and the driving base 31 is fixed at a position adjacent to the open end of the retaining base 22.

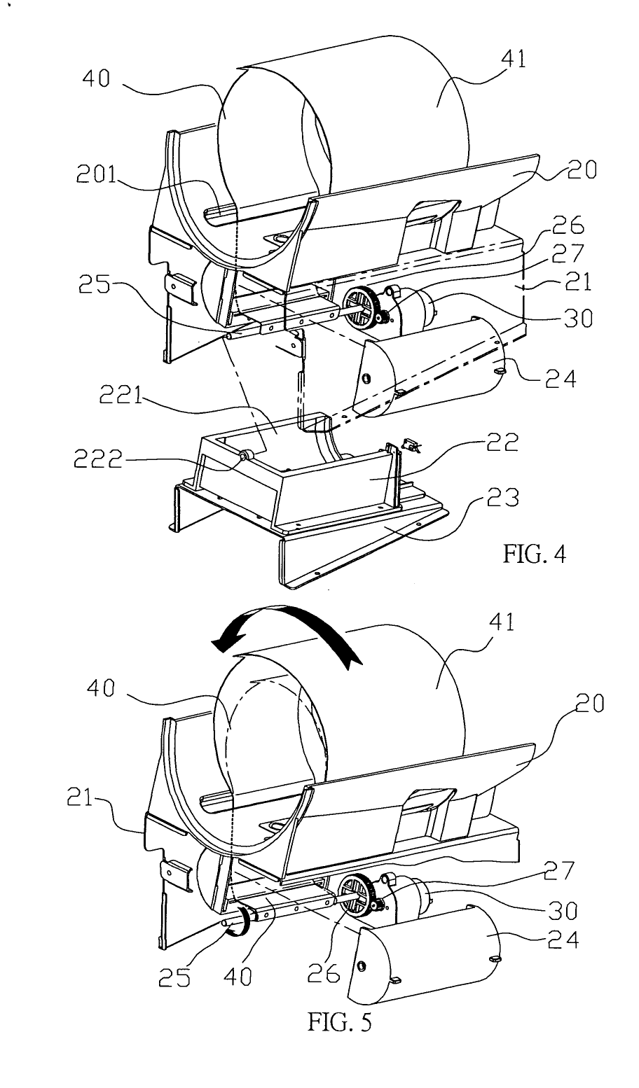

The platform 20 further comprises a penetrating hole 201 corresponding to the opening 211 of the support frame 21, a deformable and circular plate 40, and an air cuff 41 wrapped around the plate 40. One end of the plate 40 is fixed onto the platform 20 and the other end passes through the penetrating hole 201, the opening 211 and the gap of the cylinder 24 to couple with the transmission axle 25 as shown in FIG. 4 and the air cuff covers the surface of the plate 40 (not shown in the figure).

Please refer to FIGS. 2 to 5 for the use of the tunnel type sphygmomanometer according to the present invention. When the user's arm is placed in the air cuff 41 and start the start button, the control device will start the air pump and inflate the air cuff 41 through an air duct and also start the driving member 30. The driving member 30 will immediately drive the active gear 27, and the active gear 27 will drive the engaged passive gear 26 to rotate the transmission axle 25 that is coupled with the passive gear 26 as to gradually coil the plate 40 of the transmission axle 25 onto the transmission axle 25. Since the transmission axle 25 is disposed under the platform 20, therefore the plate 40 moves in the vertical direction and provides a smaller size to the sphygmomanometer. The present invention is different from the traditional sphygmomanometers pulling the plate 40 along the horizontal direction and having a larger size for the traditional sphygmomanometer.

In summation of the above description, the present invention herein enhances the performance than the conventional structure and further complies with the patent application requirements and is submitted to the Patent and Trademark Office for review and granting of the commensurate patent rights.

While the invention has been described by way of examples and in terms of preferred embodiments, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. An improved tunnel type sphygmomanometer assembly, comprising:
a platform, including a penetrating hole thereon;
a support frame, being disposed under said platform and having an opening corresponding to said penetrating hole;
a driving member, being disposed in said support frame and coupled to a transmission axle under said opening for driving said transmission axle to rotate; and
a plate, being disposed on said platform and coiled into a circular shape and said plate being deformable, and one end of said plate being fixed onto said platform and the other end passing through said penetrating hole and said opening to coupled with said transmission axle;
thereby when said driving member drives said transmission axle to rotate, said plate moves vertically downward without producing a horizontal pulling force.

2. The improved tunnel type sphygmomanometer assembly of claim 1, wherein said transmission axle is coupled to an active gear and a passive gear and said active gear and passive gear are engaged with each other.

3. The improved tunnel type sphygmomanometer assembly of claim 1, wherein said driving member is a motor.

4. The improved tunnel type sphygmomanometer assembly of claim 1, wherein said transmission axle is wrapped in a cylinder.
